(19) United States
(12) Patent Application Publication
(10) Pub. No.: US 2023/0001083 A1
(43) Pub. Date: Jan. 5, 2023

(54) ACTUATABLE DEVICE FOR PULSATILE DRUG DELIVERY

(71) Applicants: Massachusetts Institute of Technology, Cambridge, MA (US); The Brigham and Women's Hospital, Inc., Boston, MA (US)

(72) Inventors: Carlo Giovanni Traverso, Newton, MA (US); Jason Siu-Wei Li, Cambridge, MA (US); Rebecca McManus, Hingham, MA (US); Seungho Lee, Arlington, MA (US); Adam Wentworth, Rochester, MN (US); Qianqian Wan, Toronto (CA)

(73) Assignees: Massachusetts Institute of Technology, Cambridge, MA (US); The Brigham and Women's Hospital, Inc., Boston, MA (US)

(21) Appl. No.: 17/782,973

(22) PCT Filed: Dec. 4, 2020
(86) PCT No.: PCT/US2020/063318

§ 371 Date: Jun. 6, 2022

Related U.S. Application Data
(60) Provisional application No. 63/044,321, filed on Jun. 25, 2020.
(60) Provisional application No. 62/944,631, filed on Dec. 6, 2019.

(51) Int. Cl. A61M 5/142; A61M 5/145; A61M 5/168

(52) U.S. Cl. CPC A61M 5/14276; A61M 5/1452; A61M 5/16881

(57) ABSTRACT

Delivery systems and their methods of use for delivering one or more therapeutic compounds to a user are described. A delivery system may include an implantable pump and an external actuator. The implantable pump may include an actuatable portion that may be actuated to dispense a volume of fluid. The external actuator may include a pusher configured to deform skin to engage an actuatable portion of the pump. The external actuator may also include a positioning magnet configured to magnetically attract a portion of the implantable pump such that the external actuator and the pump may be aligned prior to applying force to the portion of the skin between the external actuator and the actuatable portion of the pump.

### CROSS-REFERENCE TO RELATED APPLICATIONS

This application claims the benefit under 35 U.S.C. § 119(e) of U.S. provisional application Ser. Nos. 62/944,631, filed Dec. 6, 2019, and 63/044,321, filed Jun. 25, 2020, the disclosures of which are incorporated by reference in their entireties.

### FIELD

Disclosed embodiments are related to actuated devices for pulsatile drug delivery, as well as related methods of use.

### BACKGROUND

The timing of drug administration in disease therapy may affect efficacy of medical treatments. Therefore, many drug therapies may be altered by understanding the times of the day the drugs are most effective, an approach called chronotherapy. In chronotherapy, taking medications at a specific time of day may make the therapy more effective or reduces toxic side effects depending on the circadian clock, which drives a biological rhythm of behavior and physiology with a periodicity of approximately 24 hours. However, only about 50% of individuals with chronic diseases follow their treatment regimen, making efficacy of additional timing instructions dubious. In addition, conventional controlled-release delivery systems, such as nano-particles, micro-particles, hydrogels, and microneedle patch systems, cannot deliver drugs only at the desired time over a long period. Rather, such conventional systems employ diffusion-based continuous drug release that is inappropriate in chronotherapy.

### SUMMARY

In some embodiments, an implantable pump of a therapeutic compound delivery system includes a housing, a reservoir, an actuatable portion of the implantable pump, and an actuating piston coupled to the actuatable portion of the implantable pump. The actuating piston is physically accessible through a hole formed in the housing, and the actuating piston is configured to move between a first unactuated position and a second actuated position to actuate the actuatable portion of the implantable pump to dispense a first volume from the reservoir in response to a physical force applied through the hole.

In some embodiments, an external actuator for an implanted pump includes a driveshaft movable between a first unactuated position and a second actuated position, a pusher disposed on a distal portion of the driveshaft, where the pusher is configured to apply force to skin underlying the actuator to physically actuate an implanted pump when the driveshaft is moved from the first unactuated position to the second actuated position, and a magnetic material configured to magnetically attract the external actuator toward a portion of the implanted pump so that the external actuator is aligned with a corresponding actuatable portion of the implanted pump.

In some embodiments, a therapeutic compound delivery system includes an implantable pump having a housing, a reservoir, an actuatable portion of the implantable pump, and an actuating piston coupled to the actuatable portion, where the actuating piston is physically accessible through a hole formed in the housing, and where the actuating piston is configured to move between a first unactuated position and a second actuated position to actuate the actuatable portion to dispense a first volume from the reservoir through the outlet in response to a physical force applied through the hole. The therapeutic compound delivery system also includes an external actuator having a pusher configured to apply a force to skin underlying the pusher to deform the skin into the hole of the implantable pump to actuate the actuatable portion of the implantable pump, and a first magnetic material configured to magnetically attract the external actuator toward a portion of the implantable pump so that the pusher is aligned with the actuating piston.

In some embodiments, a method of operating a therapeutic compound delivery system includes positioning an external actuator over skin under which an actuatable pump is disposed, axially aligning an actuating piston disposed in the pump with a pusher of the external actuator, and actuating the external actuator to depress the skin with the pusher to apply force to the actuating piston to operate the pump.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a side schematic of one embodiment of an implantable pump;

FIG. 2 is a side schematic of one embodiment of an external actuator;

FIG. 3A is a simplified schematic of the external actuator of FIG. 2 in use with the implantable pump of FIG. 1 in a first state;

FIG. 3B is a simplified schematic of the external actuator of FIG. 2 in use with the implantable pump of FIG. 1 in a second state;

FIG. 4A is a schematic of the external actuator of FIG. 2 in use with the implantable pump of FIG. 1 in a first state;

FIG. 4B is a schematic of the external actuator of FIG. 2 in use with the implantable pump of FIG. 1 in a second state;

FIG. 5 is a schematic of the external actuator of FIG. 2 in use with the implantable pump of FIG. 1 in a human subject;

FIG. 6 is a flow chart for one embodiment of a method of operation of a therapeutic compound delivery system;

FIG. 7 is a block diagram of one embodiment of another embodiment of an external actuator;

FIG. 8 is a flow chart for another embodiment of a method of operation of a therapeutic compound delivery system;

FIG. 9 is a graph depicting experimental results for an exemplary therapeutic compound delivery system according to exemplary embodiments described herein;

FIG. 10 is a graph depicting experimental results of an exemplary therapeutic compound delivery system according to exemplary embodiments described herein;

FIG. 11 is a graph depicting experimental results of an exemplary therapeutic compound delivery system according to exemplary embodiments described herein;

FIG. 12 is a graph depicting experimental results of an exemplary therapeutic compound delivery system according to exemplary embodiments described herein; and

FIG. 13 is a graph depicting experimental results of an exemplary therapeutic compound delivery system according to exemplary embodiments described herein.

### DETAILED DESCRIPTION

Chronotherapy is a promising component of treatments for various medical conditions. However, conventional devices are currently unable to reliably and consistently deliver a dose of a therapeutic drug at a predetermined time. Most conventional systems employ controlled-release, in which an entire dose of a drug is released over time continuously. Such conventional systems may be ineffective for providing chronotherapy for certain medical conditions, such as diabetes. Additionally, some conventional system for chronotherapy employ implanted batteries or controllers which may require frequent replacement and make the implantable device bulky and uncomfortable for a user. Some conventional systems are also not refillable, meaning that consistent delivery of the drug over long time periods is difficult and may require replacement of the delivery device. Finally, some conventional systems are magnetically or light activated, which may not be easily operable in all environmental conditions and may be interfered with by other medical equipment.

In view of the above, the inventors have recognized the benefits of a therapeutic compound delivery system that is effective for chronotherapy and is user-friendly. In particular, the inventors have recognized the benefits associated with an implantable pump that does not include an internal energy source, and an external actuator configured to operate the implantable pump. The external actuator is configured to apply physical force through the intact skin of a user to apply an actuation force to a corresponding portion of the implanted pump to actuate the implantable pump and dispense a volume of fluid. In some embodiments, the portion of the implantable pump to which the actuation force is applied may be configured in a variety of ways to avoid inadvertent physical actuation of the system as detailed further below. Such a physical actuation mechanism may be well suited to a number of different environmental conditions.

In addition to the physical actuation of an implanted pump, the inventors have also recognized the benefits associated with using magnetic materials associated with an implanted pump and associated external actuator for positioning the devices relative to one another for actuation. For example, in one embodiment, a first magnetic material such as a positioning magnet may be included in the external actuator. This first magnetic material may be magnetically attracted to a corresponding second magnetic material included in a portion of the implanted pump so that the external actuator may be aligned with an actuatable portion of the implanted pump when the first and second magnetic materials are located proximate to one another through an intervening layer of intact skin of the user. Such a therapeutic compound delivery system may be used to effectuate chronotherapy by delivering timed dosages of a drug while reducing the implanted size of the pump relative to conventional systems, though embodiments in which the disclosed systems and methods are not used for chronotherapy applications are also contemplated.

In some embodiments, an implantable pump of a therapeutic compound delivery system includes a housing, a reservoir, an actuatable portion, and an actuating piston. The housing may at least partially surround the reservoir, actuatable portion of the pump, and the actuating piston. The actuatable portion of the pump is fluidly connected to the reservoir, and is configured to dispense a predetermined volume of fluid when the actuatable portion of the pump is actuated. The actuating piston may be slidably disposed in the housing, and may be movable between a first unactuated position and a second actuated position. As the actuating piston moves from the first unactuated position to the second actuated position, the actuating piston actuates the actuatable portion of the pump to dispense the predetermined volume of fluid from the pump. The actuating piston may be physically accessible through a hole formed in the housing of the implantable pump. The pump may be implantable near the skin surface of a user, such that skin and any other intervening tissue may be deformed through the hole formed in the housing of the pump. Accordingly, the actuating piston may be movable in response to physical force applied to the skin, where the skin layer transmits the physical force to the actuating piston. As force is applied to the skin, the actuating piston may be moved linearly toward the actuatable portion of the pump and apply the physical force to the actuatable portion of the pump to dispense the predetermined volume of fluid.

According to exemplary embodiments described herein, a housing of an implantable pump includes a hole through which skin and other intervening tissue may be deformed. This physical force and deformation applied to the skin may be transferred to an actuating piston, or other actuation mechanism, disposed in the housing of the implantable pump through the hole. In some embodiments, the hole may be sized and shaped such that skin and other intervening tissue may be deformed through the hole without accidental presses, normal human contact, or other incidental forces applied to the user's body actuating the implantable pump. Accordingly, in some embodiments the hole may have a diameter or other maximum transverse dimension that is greater than or equal to 0.1 mm, 0.3 mm, 0.5 mm, 0.7 mm, 1 mm, 1.5 mm, 2 mm, 3 mm and/or any other appropriate diameter. Correspondingly, the hole may have a diameter or other maximum transverse dimension that is less than or equal to 5 mm, 4 mm, 3 mm, 2 mm, 1.5 mm, 1 mm, 0.7 mm, 0.5 mm, and/or any other appropriate diameter. Combinations of the above ranges are contemplated, including, but not limited to, diameters or another maximum transverse dimension that is between or equal to 0.1 mm and 5 mm, 0.3 mm and 3 mm, 0.3 mm and 1.5 mm, 0.5 mm and 5 mm, 1 mm and 4 mm, and/or any other appropriate combination. Of course, a hole through which skin may be deformed may have any suitable maximum transverse dimension both greater and less than the ranges noted above and/or may have a non-circular shape, as the present disclosure is not so limited.

In some embodiments, an actuatable portion of an implantable pump may include a flexible tube. The flexible tube may be fluidly connected to a reservoir by a first check valve (e.g., one way valve) configured to allow fluid to flow from the reservoir to the flexible tube, but not vice versa. The flexible tube may be coupled to a fluid outlet with a second check valve configured to allow fluid to flow from the flexible tube to the fluid outlet. The flexible tube may be configured to contain a predetermined volume of fluid to be dispensed into the body when the actuatable portion of the pump is actuated. An actuating piston disposed adjacent the flexible tube may move between a first unactuated position and a second actuated position. In the first unactuated position, the flexible tube may be in a resting position, where the flexible tube contains the predetermined volume of fluid to be dispensed. When the actuating piston is moved to the second actuated position (e.g., under application of physical force applied to skin to deform skin through a hole of an implantable pump housing) the actuating piston may apply force to deform and compress the flexible tube. As the flexible tube is compressed, the fluid may be forced out of the fluid outlet via the second check valve, as the fluid is unable to flow back into the reservoir. Accordingly, this actuation may dispense a predetermined volume of fluid into the body of a user. When the force is removed from the skin of a user and the actuating piston is free to return to the first unactuated position, the flexible tube may elastically return to the resting position where it may an initial unactuated configuration. The return of the flexible tube to the resting position draws in a second predetermined volume of fluid into the flexible tube from the reservoir. Additionally, the elasticity of the flexible tube may bias the actuating piston toward the first unactuated position. Of course, the actuating piston may be biased to the young actuated position using any appropriate method including, but not limited to, one or more separate springs, the noted elasticity of the flexible tube, and/or any other appropriate construction as the present disclosure is not limited in this regard.

According to exemplary embodiments described herein, an actuating piston of an implantable pump may move between a first unactuated position and a second actuated position to operate an actuatable portion of a pump. For example, as discussed previously, the actuating piston may move linearly to apply force to a compressible, flexible tube to dispense a predetermined volume of fluid. The amount of movement of the piston may at least partially determine the amount of fluid dispensed from the actuatable portion of the pump, and may also help to avoid inadvertent actuation of the pump . In some embodiments, a linear distance between the first unactuated position and the second actuated position of the actuating piston may be greater than or equal to 0.3 mm, 0.5 mm, 0.8 mm, 1 mm, 1.5 mm, 2 mm, and/or any other suitable distance. Correspondingly, a linear distance between the first unactuated position and the second actuated position may be less than or equal to 3 mm, 2 mm, 1.5 mm, 1 mm, 0.8 mm, 0.5 mm, and/or any other suitable distance. Combinations of the above ranges are contemplated, including a linear actuation distance of an actuating piston that is between or equal to 0.3 mm and 3 mm, 0.8 mm and 1.5 mm, 0.5 mm and 1 mm, 1 mm and 2 mm, as well as 0.8 mm and 3 mm. Of course, the actuating piston may move any suitable distance including distances both greater than and less than those noted above, as the present disclosure is not so limited.

In some embodiments, an actuating piston of an implantable pump may be formed of a magnetic material. That is, the actuating piston may be a magnet or a material that can be attracted to a magnet such as a ferromagnetic material like iron, magnetic steel alloys, or any other magnetic material. Accordingly, the actuating piston may be attracted to an appropriate corresponding magnetic materials. As will be discussed further below, the actuating piston may be used to align an external actuator with the implantable pump. For example, a magnet disposed in the external actuator may be attracted to the actuating piston. Alternatively, in some embodiments, the actuating piston may be a magnet. Accordingly, the actuating piston may be magnetically attracted to a corresponding magnet or magnetic material in the external actuator. In either case, a user may reliably align the external actuator with a corresponding portion of the implanted pump through the skin based on the magnetic attraction of the external actuator toward the actuating piston or other portion of the implanted pump arranged to appropriately position the external actuator with the implanted pump.

In some embodiments, an implantable pump includes a port configured to allow a reservoir of the pump to be refilled. The port may be formed as a septum configured to be positioned adjacent a user's skin. The septum may allow a syringe to fluidly couple to the reservoir through the skin such that a therapeutic compound may be injected into the reservoir through the skin to replenish a supply of the therapeutic compound in the reservoir. Of course, any suitable port for allowing an external volume of fluid to be inserted into a reservoir of the implantable pump may be employed, as the present disclosure is not so limited. Accordingly, the implantable pump of exemplary embodiments described herein may be refilled so that multiple doses may be delivered over a long period of time. Of course embodiments in which an implantable pump is not refillable are also contemplated.

According to exemplary embodiments described herein, an implantable portion of a therapeutic compound delivery system (e.g., a pump) may have a small size relative to conventional devices, such that the implanted device is unobtrusive to the user. In part, the small size of the implantable portion of the therapeutic compound delivery system may be attributed in part to the lack of an integrated energy source in the implantable portion. That is, the implantable portion of the therapeutic compound delivery system may be unpowered and lack a battery, though embodiments in which the various implantable systems described herein include a power source are also contemplated. In some embodiments, an implantable portion of a therapeutic compound delivery system (e.g., an implantable pump) is formed as a cylinder having an outer diameter or other maximum transverse dimension perpendicular to a longitudinal axis of the implanted device that is greater than or equal to 1 mm, 2 mm, 3 mm, 4 mm, 5 mm, and/or any other appropriate diameter. Correspondingly, the implantable portion may have an outer diameter or other maximum transverse dimension that is less than or equal to 8 mm, 6 mm, 5 mm, 4 mm, 3 mm, and/or any other appropriate diameter. Combinations of the above-noted ranges are contemplated, including an outer diameter or other maximum transverse dimension that is between or equal to 1 mm and 8 mm, 3 mm and 5 mm, 4 mm and 6 mm, as well as 1 mm and 5 mm. In some embodiments, the implantable portion has an overall length in a direction parallel to a longitudinal axis of the device that is greater than or equal to 25 mm, 30 mm, 40 mm, 45 mm, 50 mm, and/or any other appropriate length. Correspondingly, the implantable portion may have an overall length less than or equal to 75 mm, 65 mm, 60 mm, 50 mm, 45 mm, 40 mm, and/or any other appropriate length. Combinations of the above noted ranges are contemplated, including overall lengths between or equal to 25 and 75 mm, 30 and 50 mm, 45 and 50 mm, as well as 25 and 75 mm. Of course, an implantable portion of a therapeutic compound delivery system may have any suitable shape and exterior dimensions including dimensions both greater than and less than those noted above, as the present disclosure is not so limited.

In some embodiments, a therapeutic compound delivery system includes an external actuator for actuating an implantable pump of the therapeutic compound delivery system. The external actuator may be manually held on a skin surface, may be worn by a user, or may otherwise be positioned proximate to a desired portion of the user's skin such that the external actuator may apply physical force to the user's skin to operate an actuatable portion of the implantable pump of the therapeutic compound delivery system. The external actuator may include a driveshaft, a pusher, and a positioning magnet. The driveshaft and pusher may be configured to apply force to the skin of a user, with the driveshaft movable between a first unactuated position and a second actuated position. The pusher may be disposed on a distal end of the driveshaft, and the first unactuated position may be a proximal position while the second actuated position is a distal position. Accordingly, when the driveshaft is moved to the second actuated position, the pusher may apply a physical force to a user's skin when the external actuator is positioned adjacent the skin. Again, in some embodiments, a positioning magnetic material may be configured to magnetically attract the external actuator toward a portion of the implanted pump so that the external actuator is aligned with an actuatable portion of the implanted pump. In some embodiments, the positioning magnetic material may be coaxial with the driveshaft, and may be configured to magnetically attract the driveshaft toward axial alignment with an actuating piston associated with the implanted pump. In some embodiments, the positioning magnetic material may be positioned on or otherwise coupled with the driveshaft such that the positioning magnetic material may move with the driveshaft between the first unactuated position and second actuated position.

In some embodiments, an external actuator of a therapeutic compound delivery system may include an energy source, an electromechanical actuator, and a processor configured to control the electromechanical actuator. The electromechanical actuator may be coupled to the driveshaft of the external actuator and may be configured to move the driveshaft between the first unactuated position and the second actuated position. The electromechanical actuator may be any suitable actuator, including, but not limited to, a DC motor, stepper motor, or solenoid. The processor may be configured as any suitable processor with associated non-transitory processor readable memory including instructions stored in memory that when executed perform the various methods described herein. The processor may be electrically connected to the electromechanical actuator, so that the processor may control the operation of the actuator. The energy source may be a battery or another suitable electrical energy source. In some embodiments, the external actuator may be worn by a user. According to this embodiment, the processor may be configured to operate the electromechanical actuator at predetermined intervals. Accordingly, the external actuator may be employed to deliver a predetermined volume of medicinal fluid from an implanted pump at one or more predetermined intervals. In some embodiments, the timing and other characteristics (e.g., volume, flow rate, etc.) of the dispensing of the fluid may be altered. Accordingly, the external actuator may effectuate chronotherapy for a variety of therapeutic compounds.

While an embodiment in which a delivery system is used for delivering a therapeutic compounds for use in chronotherapy as described above and in relation to other embodiments herein, the current disclosure is not limited in this fashion. For example, the various embodiments described herein may be used for manual actuation, continuous delivery of therapeutic compounds, and/or any other desired application. Accordingly, the current disclosure should not be limited to only use in chronotherapy based applications.

To help determine whether or not an implanted pump and an associated external actuator are appropriately positioned relative to one another, in some embodiments, an external actuator may include a Hall Effect sensor electrically connected to a processor. The Hall Effect sensor may be configured to detect and/or measure a combined magnetic field near the external actuator. As noted previously, the external actuator may include a positioning magnetic material configured to attract the external actuator toward alignment with an actuatable portion of an implantable pump. The Hall Effect sensor may determine when the external actuator is aligned with the actuatable portion of the implantable pump based on a change in the magnetic field. In some embodiments, the implantable pump may include an actuating piston that includes a magnet, so that the Hall Effect sensor may better detect the proximity and alignment of the external actuator with the implanted pump.

In some embodiments, it may be desirable to determine whether or not an implanted pump has been appropriately actuated by an associated external actuator. In such an embodiment, an external actuator may include a force sensor configured to sense the force applied by a pusher of the external actuator to the skin of the user underlying the pusher. For example, a force sensor may be positioned between a pusher and a driveshaft electrically connected to a processor though other force sensing arrangements may also be used. The force sensor may be configured to measure a force applied by the driveshaft to an adjacent surface (e.g., skin). Accordingly, the force sensor may be employed to determine if a threshold force has been applied by the external actuator to the adjacent surface. In some cases, the threshold force may correspond to a volume of fluid dispensed from an implanted pump, a pusher not being aligned with an actuatable portion of an associated implanted pump, or other appropriate operating status. Accordingly, if the threshold force is not reached, the external actuator may be operated again to try and actuate the system with a force greater than or equal to the threshold force.

In some embodiments, an external actuator may include an accelerometer configured to measure acceleration of the external actuator, and the accelerometer may be electrically connected to a processor. The information from the accelerometer may be employed to determine an orientation of the external actuator or a circadian rhythm of the patient. That is, the information from the accelerometer may be used to determine an awake or sleeping status of the patient, such that the external actuator may be operated to dispense a volume of fluid at a time appropriate for a given chronotherapy. Of course, the information from the accelerometer may be employed for any suitable purpose, as the present disclosure is not so limited.

In some embodiments, an external actuator may include a trigger operative coupled to the driveshaft so that the driveshaft may be manually moved between a first unactuated (e.g., proximal) position and a second actuated (e.g., distal) position. In some embodiments, the external actuator may be used by a user even when the driveshaft is connected to an electromechanical actuator. Accordingly, in this embodiment, a user may manually dispense a volume of fluid using the external actuator regardless of an energy state of the external actuator. Of course, in other embodiments an external actuator may not be manually operable, as the present disclosure is not limited in this regard.

In some embodiments, a therapeutic compound delivery system may include both an implantable pump having an actuatable portion, as well as an external actuator. The implantable pump may be implantable beneath a skin layer of a user, while the external actuator is placed against the outside of the skin. The external actuator may be configured to be removable from the user, or may be configured to be worn. Accordingly, the external actuator may include an elastic strap, hook and loop fastener strap, cinch strap, adhesive, be incorporated into a garment worn by the user, and/or may incorporate any other suitable arrangement for securing the external actuator to the body of a user. Of course embodiments in which the external actuator is not held in place on the body of a user when not in use are also contemplated.

In some embodiments, an external actuator of a therapeutic compound delivery system may communicate with one or more remote devices, including, but not limited to, a mobile device (e.g., smartphone), personal computer, and remote server (e.g., cloud computing service). The remote device may include a graphical user interface (e.g., as a part of an application) through which a user or medical professional may generate commands to the external actuator. That is, the graphical user interface may be used to alter one or more operating characteristics of the external actuator during a drug delivery process based on user input. For example, the graphical user interface may be employed to alter one or more of daily maximal dosage, delivery interval, dosage mode (e.g., single dosage), start time, or another suitable characteristic. In this manner, the external actuator may be easily controlled by a user using any appropriate computing device. Of course, in other embodiments an external actuator may not communicate with a remote device, as the present disclosure is not so limited.

As noted above, the various embodiments described herein may include one or more therapeutic compounds disposed within a reservoir of an implanted pump. However, it should be understood that the current disclosures are not limited to use with any particular type of therapeutic compound. Thus, therapeutic compounds for purposes of this application may correspond to any appropriate material including, but not limited to, any drug, medication, pharmaceutical preparation, contrast agent, and/or biologic such as a protein, antisense molecule, and gene therapy viral vector as the disclosure is not so limited. When a therapeutic compound delivered using the systems and methods described herein is present in a particular location in an “effective amount” it means a concentration of the therapeutic compound is greater than or equal to a trace amount and is sufficient for achieving a desired purpose, such as, for example, to permit detection of the therapeutic compound in a subject for diagnostic purposes, to treat a disease or condition in a subject, and/or enhance a treatment of a disease or condition in a subject. In some embodiments, an effective amount of a particular therapeutic compound is present in an amount sufficient to reduce or alleviate one or more conditions associated with a particular condition.

For the purposes of this disclosure, a magnetic material may correspond to any material that is attracted to a magnetic field and/or that is capable of attracting another material with a magnetic field emitted by the magnetic material. Thus, appropriate combinations of magnets and materials that may be magnetized such as ferromagnetic materials and paramagnetic materials may be used. For example, two appropriately oriented magnets, a magnet and a material that may be magnetized, and combinations of the foregoing may be used to provide the desired magnetic properties between the various portions of a system. Further, it should be understood that the disclosure is not limited to which of the components includes a magnet. Thus, the various embodiments described herein may include any appropriate combination of magnetic materials to provide the desired functionalities as the disclosure is not limited in this fashion. Though instances in which the embodiments described herein do not include magnetic materials are also contemplated.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

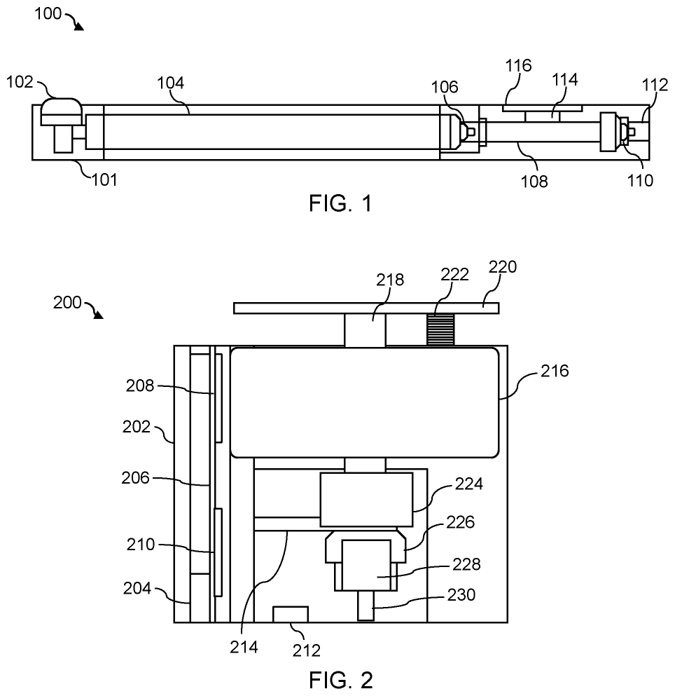

FIG. 1 is a side schematic of one embodiment of an implantable pump 100. As shown in FIG. 1, the implantable pump includes a housing 101, which in the current embodiment has a cylindrical shape. As shown in FIG. 1 the implantable pump includes a raised septum 102. The septum is configured to allow a syringe or other device to fluidly connect to the implantable pump through the septum to allow the pump to be refilled. Additionally, the raised septum may simplify locating and refilling the implantable pump. Of course, in other embodiments the septum may not be raised as the present disclosure is not so limited. The septum is coupled to a reservoir 104 which extends along a majority of a length of the housing 101. The reservoir may include a therapeutic compound in the of slurries, emulsions, liquids, and/or other appropriate forms disposed therein. The reservoir is fluidly coupled to an actuatable portion of the pump including a volume defined by a flexible tube 108 via a first check valve 106. The first check valve 106 allows flow from the reservoir to the flexible tube 108 while preventing a backflow from the flexible tube into the reservoir. The flexible tube is fluidly connected to an outlet 112 on an end opposite the first check valve 106. The flexible tube is connected to the outlet 112 via a second check valve 110, which allows a therapeutic compounds to flow from the flexible tube to the outlet while preventing backflow from the outlet to the interior volume of the flexible tube. The outlet is arranged to allow a therapeutic compound disposed in the reservoir to be delivered subcutaneously upon actuation of the implantable pump. Of course, while a particular construction of an actuatable portion of an implantable pump has been described above, it should be understood that an implantable pump may include any appropriate construction capable of actuation using the methods and systems disclosed herein as the disclosure is not limited to the specific type of pump used.

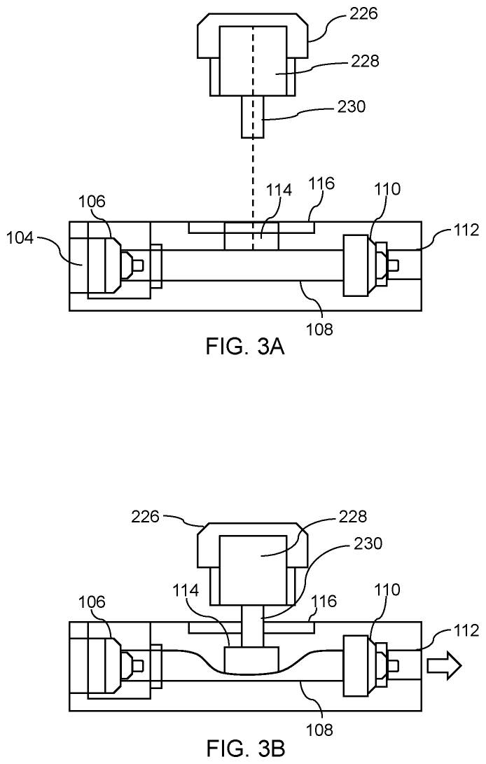

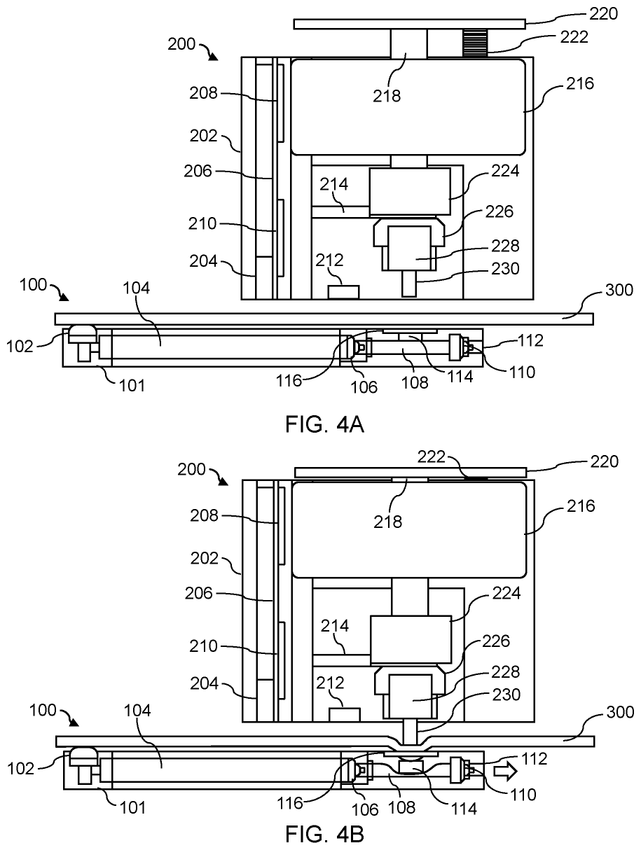

According to the embodiment of FIG. 1, the implantable pump includes an actuating piston 114 that is configured to move between a first unactuated position (shown in FIG. 1) to a second actuated position (see FIG. 3B). The actuating piston is configured to move transverse (e.g., perpendicular) to a longitudinal axis of the implantable pump housing 101 the other actuation directions are also possible. In the embodiment of FIG. 1, the actuating piston moves substantially linearly between the first unactuated position and the second actuated position. As will be discussed further with reference to FIGS. 3A-4B, the actuating piston is configured to be moveable under force applied to skin, where the force is transferred through the skin, and any other intervening tissue, to the actuating piston. The actuating piston is accessible through a hole 116 formed in the housing 101. The hole may be sized and shaped such that a skin layer (e.g., approximately 2 mm thick) may be deformed through the hole, but small enough that force application by a human finger or other incidental contact is not able to deform skin through the hole to an extent that actuates the implantable pump.

FIG. 2 is a side schematic of one embodiment of an external actuator 200. As shown in FIG. 2, the external actuator includes a housing 202 configured to at least partially contain the various components of the external actuator. In the embodiment of FIG. 2, the external actuator includes an energy source 204 in the form of a battery, which supplies electrical energy to the various components of the external actuator though other appropriate energy sources may also be used. The external actuator also includes a control circuit board 206 having a processor 208 and associated memory. The external actuator may also include an accelerometer 210, a Hall Effect sensor 212, and/or a force sensor such as a force sensitive resistor 214. The housing 202 of the external actuator houses a solenoid 216 or other actuator which is coupled to a driveshaft 218. The driveshaft is configured to move between a first unactuated position (shown in FIG. 2) and a second actuated position (see FIG. 4B). According to the embodiment of FIG. 2, the first position is a proximal position, and the second position is a distal position. The driveshaft is also coupled to a handle 220 or other trigger, which in turn is coupled to a biasing spring 222. The handle or other trigger may be configured to allow a user to manually move the driveshaft between the first position and the second position to manually actuate the external actuator. The biasing spring 222 is configured as a compression spring, and biases the driveshaft toward the first unactuated position.

As shown in FIG. 2, the driveshaft 218 is coupled to a collar 224, a positioning magnet coupler 226, a positioning magnet, and a pusher 230. The collar 224 is configured to hold the force sensitive resistor 214 between the collar 224 and the positioning magnet coupler 226, such that the force sensitive resistor is able to detect the force applied through the driveshaft. Of course, a force sensitive resistor or other stress/strain sensor may be employed to detect the force applied through the driveshaft and may be mounted in any suitable configuration, as the present disclosure is not so limited. The positioning magnet coupler 226 holds the positioning magnet in a position coaxial with the driveshaft 218. In other embodiments an alternative connection between the positioning magnet and driveshaft may be employed as well as arrangements in which the positioning magnet and/or other appropriate magnetic material are not coaxial with the driveshaft are also contemplated. The positioning magnet is configured to magnetically attract the external actuator toward a portion of an implantable pump, so that the driveshaft 218 of the external actuator may be aligned with a corresponding actuatable portion of the implantable pump. As shown in FIG. 2, a pusher 230 is coupled to the driveshaft 218 at a distal most position and is configured to apply physical force to the skin of a user to operate an implanted pump beneath the skin. The operation of the pusher 230 and driveshaft 218 will be discussed further with reference to FIGS. 4A-4B.

According to the embodiment of FIG. 2, the combination of the energy source 204, processor 208, accelerometer 210, Hall Effect sensor 212, and force sensitive resistor 214 may be used to control the operation of a corresponding implanted pump, and to verify that the implanted pump is being operated correctly to achieve a therapeutic objective. The accelerometer 210 may provide information to the processor 208 regarding a local gravitational direction and the movement of a user wearing the external actuator. This accelerometer information may be used to determine a circadian rhythm of a wearer or sleep-wake cycles to effectuate chronotherapy. For example, orientation data indicating a person is in a prone position may be associated with a sleep cycle as compared to orientation data indicating a person is in an upright position associated with a wake cycle. The Hall Effect sensor 212 may provide information regarding a local magnetic field, and in particular may detect when a magnetic material, such as the positioning magnet 228, is appropriately aligned with and proximate to a corresponding magnetic portion of an implantable pump including a corresponding magnetic material that may be attracted to the magnetic material of the external actuator. Accordingly, the Hall Effect sensor information may be used to indicate to a user that the external actuator is appropriately aligned with an implantable pump, and/or may be employed to inhibit operation of the external actuator until the external actuator is appropriately aligned with an implantable pump. The force sensitive resistor 214 is configured to measure the force applied through the driveshaft to a skin surface, and correspondingly, to an actuatable portion of an implanted pump. The information from the force sensitive resistor may be provided to the processor, and may be used to determine if a threshold force has been applied to the skin surface. The threshold force may correspond to a full actuation of the implanted pump, so that the processor may determine if a predetermined volume of fluid has been successfully dispensed from the pump. The operation of the external actuator using these various sensor inputs is detailed further below.

FIG. 3A is a simplified schematic of the external actuator of FIG. 2 in use with the implantable pump of FIG. 1 in a first state. As shown in FIG. 3A by the dashed line, the pusher 230 and positioning magnet 228 may be axially aligned with the actuating piston 114 during actuation of the implanted pump. The actuating piston 114 may be formed at least partially from a magnetic material such that the positioning magnet, or other magnetic material of the external actuator, maybe attracted toward the actuating piston. That is, when the positioning magnet and actuating piston are not coaxially aligned, magnetic attraction between the actuating piston and positioning magnet draws the positioning magnet into axial alignment with the actuating piston. As the positioning magnet and pusher 230 are coaxial, the alignment of the positioning magnet 228 relative to the actuating piston also aligns the pusher 230 with the actuating piston. Of course, other embodiments in which the positioning magnet may not be coaxial with the pusher and may instead be attracted to another magnetic portion of the implantable pump with appropriate offsets so that the pusher 230 may still be aligned with the actuating piston 114 as a result of magnetic attraction are also contemplated. In some embodiments, the pusher 230 may also include a magnetic material to assist in magnetically attracting the pusher toward alignment with the actuating piston.

According to the state shown in FIG. 3A, the actuating piston 114 is in a first unactuated position. The flexible tube 108 is correspondingly in an undeformed resting position. The pusher 230 (and associated driveshaft not shown in the current figure) is in a first unactuated position. The actuating piston 114 is accessible through a hole 116 formed in the housing of the implantable pump.

FIG. 3B is a simplified schematic of the external actuator of FIG. 2 in use with the implantable pump of FIG. 1 in a second state. As shown in FIG. 3B, the actuating piston 114 is in a second actuated position. The flexible tube 108 is correspondingly in a deformed position. That is, the actuating piston 114 has pressed against the flexible tube. As the flexible tube is depressed, a predetermined volume of fluid inside of the flexible tube is pressurized. As the first check valve 106 prevents flow in a direction of the reservoir, the predetermined volume of fluid is forced out of the fluid outlet 112 through the second check valve 110, as shown by the arrow. To move the actuating piston to the second actuated position, the pusher 230 has been moved to a second actuated position. In the simplified schematic of FIGS. 3A-3B, skin is not shown between the external actuator and the implantable pump. Accordingly, the pusher abuts the actuating piston to apply physical force to the actuating piston 114 toward the flexible tube. However, when the implantable pump is implanted, a skin layer would be positioned between the pusher 230 and the actuating piston 114, with the skin deformed through the hole 116. Regardless, direct or indirect physical force transmission between the pusher 230 and actuating piston 114 moves the actuating piston to the second actuated position. According to the embodiment of FIGS. 3A-3B, the elasticity of the flexible tube 108 may bias the actuating piston 114 toward the first unactuated position. In particular, the resiliency of the flexible tube may resist the movement of the actuating piston from the first unactuated position to the second actuated position. Of course, in other embodiments any suitable method for biasing the piston or other portion of an implantable pump to (e.g., a spring) toward the first unactuated position may be used as the present disclosure is not so limited. When the actuating piston is allowed to move back to the first unactuated position, the flexible tube may return to the undeformed and/or initial configuration. As the flexible tube returns to the initial configuration, a second predetermined volume of fluid may be drawn from the reservoir through the first check valve 106 into the flexible tube. Accordingly, once the actuating piston returns to the first unactuated position, the implantable pump may be prepared to dispense another predetermined volume of fluid.

FIGS. 4A-4B are schematics of the external actuator 200 of FIG. 2 in use with the implantable pump 100 of FIG. 1 in a first on actuated state and second actuated state, respectively. According to the state shown in FIG. 4A, the driveshaft 218 of the external actuator is in a first unactuated position. Accordingly, the pusher 230 is retracted inside of the housing 202, so that the housing may be placed flush against a user's skin 300. Of course, in some embodiments the pusher 230 may not be retracted inside of the housing 202, and the housing need not be placed flush against the user's skin for operation. As shown in FIG. 4A, the actuating piston 114 is in a first unactuated position. Correspondingly, the flexible tube 108 is undeformed and contains a predetermined volume of fluid. The implantable pump 100 is disposed just under a skin layer 300 in a user. As shown in FIG. 4A, the septum 102 may be positioned closest to the skin layer 300 so that the septum is easy to locate for refilling the reservoir 104.

From the state shown in FIG. 4A, the processor 208 operates the solenoid 216, or other actuator, to move the driveshaft from the first unactuated position shown in FIG. 4A to the second actuated position shown in FIG. 4B. The processor 208 may wait to operate the solenoid 216 until the Hall Effect sensor detects that the positioning magnet 228 and the actuating piston 114 are aligned. Alternatively, rather than using the solenoid, a user may manually depress the handle 220 against the biasing force of the biasing spring 222 to move the driveshaft to the second actuated position. When the driveshaft is in the second actuated position, the pusher 230 projects out of the housing 202 and applies physical force to the skin layer 300. As shown in FIG. 4B, the portion of the skin layer 300 disposed between the pusher and the hole 116 may be deformed such that a portion of the skin layer, and any other intervening tissue, extends into the hole 116 formed in the implantable pump housing 101. Thus, force from the pusher 230 is transferred to the actuating piston 114 through the skin 300. Accordingly, as the actuating piston is moved to the second actuated position, a predetermined volume of fluid may be dispensed from the flexible tube 108, as discussed previously with reference to FIGS. 3A-3B. The actuation process may be repeated as necessary to dispense a given volume of fluid over a desired time period. In some embodiments, the reservoir 104 may be refilled so that the therapeutic compound delivery system including the external actuator and the implantable pump may be used to deliver multiple doses of a therapeutic fluid over a long period of time on the order of weeks or months.

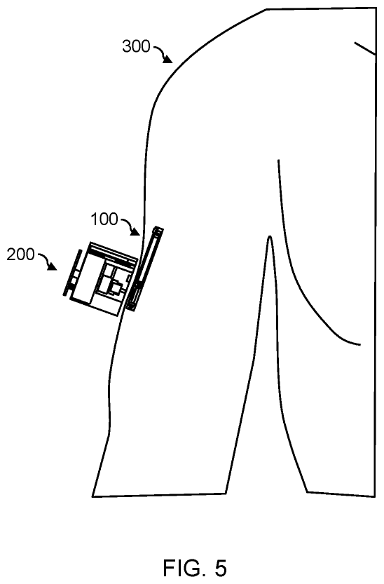

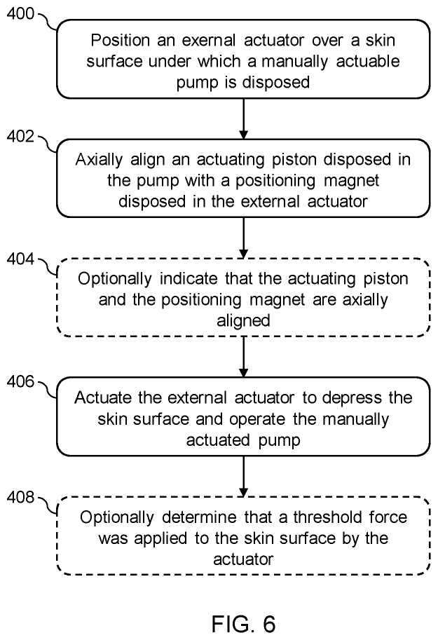

FIG. 5 is a schematic of the external actuator 200 of FIG. 2 in use with the implantable pump 100 of FIG. 1 in a human subject. As shown in FIG. 5, the implanted pump may be implanted just below a skin surface 300 of the user. In the embodiment shown in FIG. 6, the implanted pump is implanted in the upper arm or bicep region of a user. Of course, the implanted pump may be implanted at any suitable location on the human body, as the present disclosure is not so limited.

FIG. 6 is a flow chart for one embodiment of a method of operation of a therapeutic compound delivery system. At step 400, an external actuator is positioned over a portion of a user's skin under which a manually actuatable pump is disposed. At step 402, an actuating piston disposed in the pump is axially aligned with a positioning magnet disposed in the actuator. At optional step 404, the method includes indicating that the actuating piston and the positioning magnet are axially aligned. This indication may be tactile, as a user may feel when the positioning magnet is aligned based on changes in the magnetic field between the actuating piston and positioning magnet. In some embodiments, the external actuator may include a light or speaker to provide a visual or auditory indication as to the alignment state of the external actuator. In some embodiments, the indication may be relayed via a remote device (e.g., a mobile device) that communicates with the external actuator. At step 406, the actuator is actuated to depress the skin surface and operate the manually actuated pump. As discussed previously, this actuation may include operation of an electromechanical actuator (e.g., solenoid), or the actuation may be manual. Operating the manually actuated pump may include moving the actuating piston from a first unactuated position to a second actuated position. At optional step 408, a processor may receive one or more signals related to a force applied by a driveshaft of the external actuator to the underlying skin. The processor may compare the measured force to a predetermined threshold force to determine if the therapeutic compound delivery system was appropriately actuated. Based on this determination, an appropriate indication of the forced sense relative to the threshold may be output to a user using lights, sound, text, graphics, and/or any other appropriate indication. For example, if a sensed force is less than the predetermined force threshold, an indication of the sensed state may be output. This determination may inform a user whether a predetermined volume of fluid has been successfully dispensed from the implanted pump when the external actuator and implanted pump are appropriately aligned.

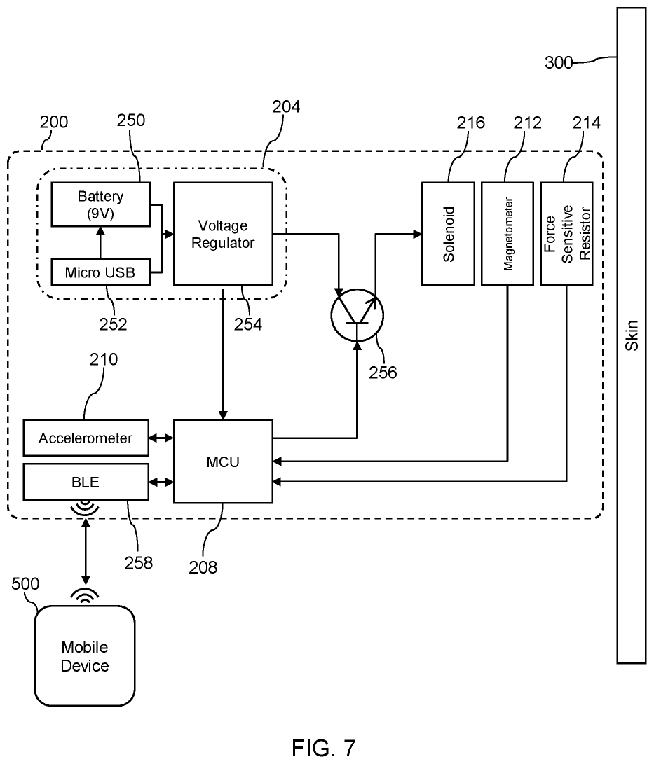

FIG. 7 is a block diagram of one embodiment of another embodiment of an external actuator 200, showing one embodiment of an electrical arrangement for an external actuator. As shown in FIG. 7, the external actuator includes an energy source 204. In the depicted embodiment, the energy source includes a battery 250, micro USB port 252, and voltage regulator 254. The battery may provide a portable source of power, and the micro USB port may allow the battery to be recharged, or alternatively provide a wired source of power to the external actuator. The voltage regulator may step down or step up the voltage from the battery and micro USB to a consistent voltage used for the rest of the components. For example, the voltage regulator may regulate the voltage to 12V, 5V, or another appropriate voltage. The energy source 204 is connected to a transistor 256 and a microcontroller unit 208 (e.g., a processor). The microcontroller unit (MCU) 208 controls the transistor and selectively allows power to flow to a solenoid 216, such that the solenoid is selectively electrically connected to the energy source 204. The MCU is also electrically connected to a magnetometer 212 (e.g., Hall Effect sensor), a force sensitive resistor 214, and an accelerometer 210, which each supply the above described sensed information to the MCU. According to the depicted embodiment, the MCU 208 is also connected to a Bluetooth Low Energy (BLE) transceiver 258. The BLE transceiver is configured to allow wireless communication with one or more remote devices, such as a mobile device 500. The mobile device may include a graphical user interface which allows a user to control certain parameters or otherwise retrieve information about the external actuator. Of course, while a BLE transceiver is employed in the depicted embodiment, any suitable wireless transceiver may be employed such as a Wi-Fi transceiver, ZigBee transceiver, radio transceiver, etc. as the present disclosure is not so limited.

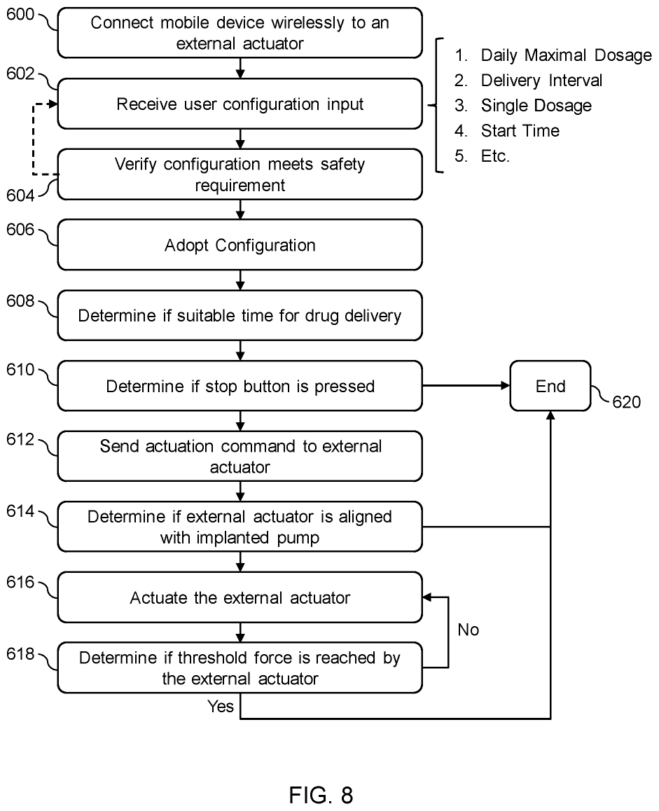

FIG. 8 is a flow chart for another embodiment of a method of operation of a delivery system. At step 600, a mobile device is wirelessly connected to an external actuator. At step 602, user configuration input is received. The user configuration input may be input by a user on a graphical user interface on the mobile device, or may be transmitted from a remote server and controlled by a medical professional. The configuration input may include, but is not limited to, daily maximal dosage, delivery interval, single dosage mode, start time, combinations of the foregoing, and/or any other appropriate operating parameter. At step 604, the configuration may be verified to make sure the input operating parameters meet one or more safety requirements including for example toxicity limits, maximum permissible dosages, maximum doses for a given time period, and/or other appropriate parameters. If the safety requirement is not met, a new configuration input may be required at step 602. At step 606, a configuration is adopted when the safety requirement is met and the configuration may be stored in memory for execution by the system processor.

As shown in FIG. 8, in some embodiments, at step 608 a system may determine if it is a suitable time for delivery of a therapeutic compound. The external actuator may perform this determination based on information from clock, accelerometer, and/or any other appropriate information source. In some embodiments, a mobile device may perform step 608. At step 610, it is determined if a stop button is pressed. In some embodiments, the stop button may be disposed on the external actuator, and may prevent actuation of the external actuator. If the stop button is pressed, the method may immediately terminate at step 620. If the stop button is not pressed, the method may proceed to step 612, where an actuation command is sent to the external actuator. At step 614, it is determined if the external actuator is aligned with an implanted pump. This determination may be based at least in part on information from a Hall Effect sensor which detected a combined magnetic field formed by the external actuator and the implanted pump. This determination may be done by comparing a sensed magnetic signal and a predetermined magnetic signal when the corresponding portions of the external actuator and implanted pump are aligned. If the external actuator is not aligned, the method may terminate at step 620. However, if the external actuator is aligned, the method may proceed to step 616, in which the external actuator is actuated. This actuation may operate the implanted pump to dispense a predetermined volume of fluid. At step 618, it may be determined if a threshold force is reached by the external actuator, which may be indicative of a successfully dispensing of the predetermined volume of fluid. If a threshold force was not reached by the external actuator, in some embodiments, the processor of the external actuator may perform another actuation cycle either until a predetermined number of actuation cycles have been reached after which an error state may be output to the operator and/or until a successful actuation has been indicated by a force greater than the predetermined threshold force has been sensed. Once a sensed force greater than the predetermined threshold force has been sensed, the actuation cycle may end. Of course, embodiments in which an applied force is not measured to determine a successful actuation cycle are also contemplated.

In some embodiments, the methods described with reference to FIGS. 5 and 8 may be repeated as necessary according to a given drug delivery schedule. For example, in some embodiments the methods may be repeated for delivering a predetermined volume of fluid at a certain interval of time. As another example, in some embodiments the methods may be repeated for delivering a predetermined volume of fluid at a given sleep or wake state. As still yet another example, the methods may be repeated to deliver a larger volume of fluid than may be provided by a single actuation of an external actuator.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device including a processor used with any of the embodiments described turn may be embodied in any of a number of forms, such as a an integrated processor, a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a tablet, a smartphone or any other suitable portable or fixed electronic device.

Also, a computing device may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a processor readable storage medium (or multiple processor readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more processors, perform methods that implement the various embodiments discussed above. As is apparent from the foregoing examples, a processor readable storage medium may retain information for a sufficient time to provide computer-executable instructions in a non-transitory form. Such a processor readable storage medium or media can be transportable, such that the program or programs stored thereon can be loaded onto one or more different computing devices to implement various aspects of the present disclosure as discussed above. As used herein, the term “processor-readable storage medium” encompasses only a non-transitory processor-readable medium that can be considered to be a manufacture (i.e., article of manufacture) or a machine. Alternatively or additionally, the disclosure may be embodied as a processor readable medium other than a processor-readable storage medium, such as a propagating signal.

The terms “program” or “software” are used herein in a generic sense to refer to any type of computer code or set of processor-executable instructions that can be employed to program a processor to implement various aspects of the present disclosure as discussed above. Additionally, it should be appreciated that according to one aspect of this embodiment, one or more programs that when executed perform methods of the present disclosure need not reside on a single computing device or processor, but may be distributed in a modular fashion amongst a number of different computing devices or processors to implement various aspects of the present disclosure .

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

### EXAMPLES

According to exemplary embodiments described herein, a delivery system may be employed to deliver dosages of a variety of therapeutic compounds. The compounds may be in liquid form, or may be otherwise suspended in a liquid solution. Examples of drug formulations that may be employed with delivery systems of exemplary embodiments described herein include bromocriptine, lisinopril, cortisol, cortisol derivatives, mineralocorticoids, growth hormone, and statin. A wide variety of solvents may be employed in the formulation of these drugs, including, but not limited to PBS, sesame oil, corn oil, soybean oil, olive oil, peanut oil, lard oil, rapeseed oil, cottonseed oil, linseed oil, sunflower seed oil, ethanol, tween 20, and ethanol, tween 80.

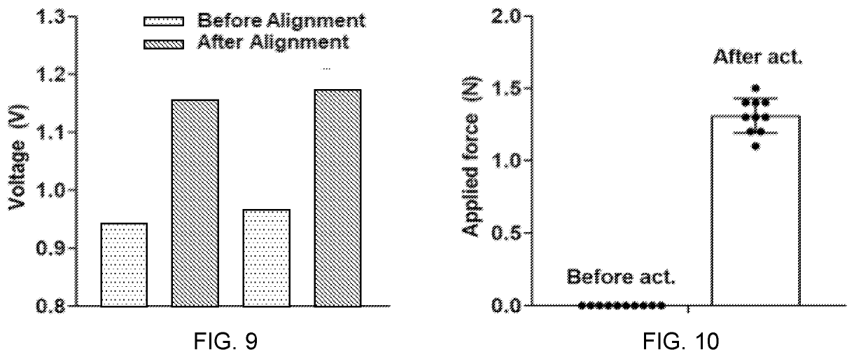

To verify the beneficial characteristics of therapeutic compound delivery systems according to exemplary embodiments described herein, experimental actuation tests were carried out, the results of which are shown in FIGS. 9-13. Firstly, the accuracy of the Hall Effect sensor was employed to determine whether the alignment may be accurately measured. In the test, the alignment was tested with a positioning magnet and an actuating piston having opposite magnetic polarities. The results of the test are shown in FIG. 9, which shows the Hall Effect sensor reliably reporting alignment with a change in sensed voltage corresponding to a predetermined voltage change and/or absolute voltage associated with the aligned position. Next, a force sensitive resistor was tested to determine whether a threshold force for dispensing a predetermined volume of fluid could be determined. The results of the tests are shown in FIG. 10, where the force applied to dispense a predetermined volume was reliably repeatable indicating a threshold value for determining appropriate actuation may be used.

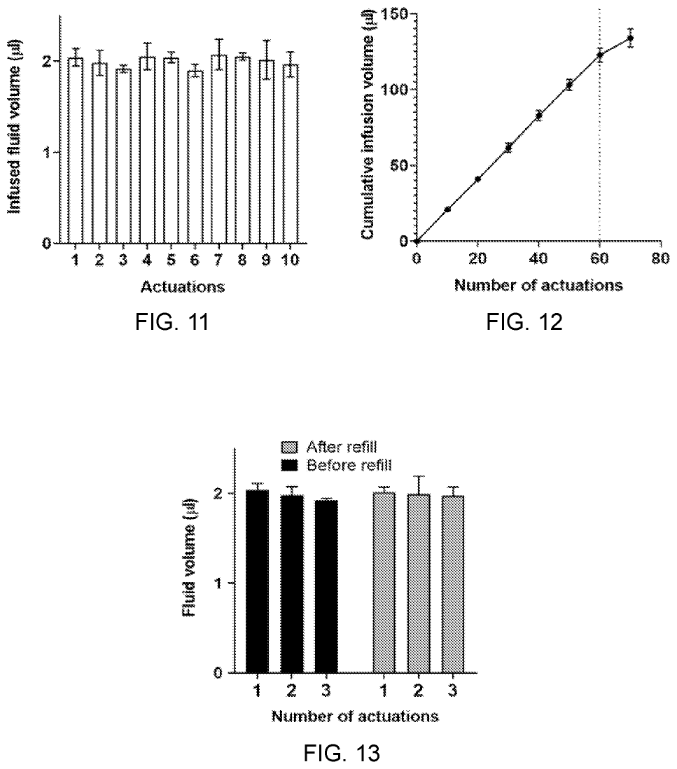

Additional experiments were determined to test the repeatability of dispensing a predetermined volume of fluid. During these experiments, an implantable pump like that of FIG. 1 was employed. The pumps filled with a bromocriptine solution (20 mg ml−1) were fully immersed in phosphate buffered saline (PBS; pH 7.4) at 37° C. and were actuated with the external actuator for ten consecutive times. That is, 20 mg of bromocriptine powder was dissolved in 0.5 ml EtOH (ethanol) and 0.5 ml Tween80. Of course, other formulations are contemplated and the amount of various drug components may be varied. In some embodiments, a ratio of the EtOH and the Tween80 need not be in a 1:1 ratio. For example, bromocriptine may be dissolved in solely EtOH up to approximately 7 mg/ml and solely Tween 80 up to approximately 5 mg/ml. In cases where EtOH and Tween 80 are used in combination, bromocriptine may be dissolved at concentrations between 0 and 23 mg/ml. The drug in the reservoir was infused like a water-jet and the infused fluid volume per actuation was 2.0±0.12 μl, indicating high reproducibility even with ten consecutive actuations, as shown in FIG. 11. Accordingly, therapeutic drug delivery systems according to exemplary embodiments described herein may generate a range of sequential doses that are within 10% of a target dosage. To evaluate long-term feasibility and to assess any possible leakage from the pump, the pump was immersed in PBS (Phosphate-buffered saline) for 28 days. The average infused fluid volume per actuation during immersion was 1.96±0.14 μl, similar to that described above, again showing high reproducibility, and no drug was detected during non-actuation periods. Due to the flexible drug reservoir, the pump showed a reproducible infusion volume per actuation of 1.93±0.12 μl with up to 60 consecutive actuations until approximately 89.1% of the total volume of the solution in the drug reservoir was consumed, as shown in FIG. 12. The effect of refilling the reservoir was also tested, with the infusion profile per actuation not being affected by the refilling procedure, as shown in FIG. 13. During these experiments, it was also shown bromocriptine and lisinopril formulation in the pump were stable without apparent denaturation when stored in the pump at 37° C. for 4 weeks.

To demonstrate the effect of chronotherapy, the therapeutic compound delivery system was experimentally employed in two different applications; one was a type 2 diabetes application using a bromocriptine that is a sympatholytic dopamine D2 receptor agonist and exerts inhibitory effects on serotonin turnover in the central nervous system. The other application was a hypertension application using a lisinopril that is an angiotensin converting enzyme (ACE) inhibitor.

First, the in vivo pharmacodynamics of bromocriptine using Zucker diabetic fatty (ZDF) rats was investigated, as a model of type 2 diabetes. For five different animal groups, the same volume of bromocriptine or bromocriptine free solution was administered daily for 4 weeks. For the parameters of pharmacodynamics, the profiles of body weight and food intake were first measured. The body weight gain and the total food intakes in all bromocriptine groups were significantly lower than those of both control groups. More importantly, both Inj-Br-E and Pump-Br-E exhibited a significantly lower body weight gain and total food intake than those treated with Inj-Br-M (P<0.05). During the intraperitoneal glucose tolerance test (IPGTT) performed after 4 weeks of treatment, all bromocriptine groups in pharmacodynamic parameters related to postprandial glucose metabolism showed better results than control groups. Significantly, the blood glucose levels after a glucose load and the area under the plasma glucose concentration-time curve (AUCglucose) of both Inj-Br-E and Pump-Br-E were lower than those of the Inj-Br-M (FIG. 3d). The plasma insulin levels during the IPGTT and AUCinsulin in both evening bromocriptine groups were comparably lower than those with the morning bromocriptine group. In addition, there was no significant difference in the above-mentioned pharmacodynamic parameters between the Inj-Br-E and Pump-Br-E groups, suggesting that the efficacy of the bromocriptine delivered with the pump was nearly identical to that administered via subcutaneous injections. The results were consistent with data obtained in many vertebrate species, including humans, which have shown that bromocriptine administration improves glucose tolerance.

To demonstrate the feasibility of high blood pressure treatments, the in vivo pharmacokinetics profiles of lisinopril with the pump and subcutaneous injection groups was evaluated. The pharmacokinetic profiles from the Pump-lisinopril and Inj-lisinopril groups were superimposable with a similar maximum plasma concentration (Cmax) of 394.2±83.2 ng ml−1 and 322.7±64.7 ng ml−1, respectively. The time taken to reach the Cmax (Tmax) was 30 min in both groups. The areas under the curve of plasma lisinopril concentration versus time (AUC) in the pump and subcutaneous injection group were 25335.5±7640.9 and 24484.5±5643.8 ng ml−1 min, respectively, which were not significantly different (p>0.05). Drug leakage in the implanted pump was not detected during the non-actuation periods over a 28-day period. The maximum plasma concentration of drug did not vary significantly, showing that the refilling procedure still did not affect the in vivo performance of the pump .

To evaluate in vivo biocompatibility of the pump, biopsied tissues samples from three different locations around the pump were examined by both H&E and Masson's trichrome (MT) staining; 1) the outlet, 2) the area for actuation (i.e., the actuation), and 3) the wall. The overall inflammatory response was observed to be minor, irrespective of tissue location. Importantly, the tissue over the actuation area did not exhibit any adverse sign after repetitive actuations. The MT-stained tissues showed the formation of a fibrous capsule around the implanted pump, where the capsule thickness was measured as 140.7±30.01 μm (FIG. 4b). The outlet was not blocked when observed at the end point of the experiments (i.e., 30 days after implantation).

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. An implantable pump of a therapeutic compound delivery system comprising:
a housing;
a reservoir;
an actuatable portion of the implantable pump; and
an actuating piston coupled to the actuatable portion of the implantable pump, wherein the actuating piston is physically accessible through a hole formed in the housing, and wherein the actuating piston is configured to move between a first unactuated position and a second actuated position to actuate the actuatable portion of the implantable pump to dispense a first volume from the reservoir in response to a physical force applied through the hole.

2. The therapeutic compound delivery system of claim 1, wherein the actuating piston comprises a magnetic material.

3. The therapeutic compound delivery system of claim 2, wherein the actuating piston is a magnet.

4. The therapeutic compound delivery system of claim 2, wherein the actuating piston is configured to magnetically attract a positioning magnet of an external actuator.

5. The therapeutic compound delivery system of claim 4, wherein the magnetic attraction between the positioning magnet and the actuating piston is configured to axially align the positioning magnet and the actuating piston.

6. The therapeutic compound delivery system of claim 1, wherein the hole is sized and shaped to receive skin pressed by an external actuator, wherein the external actuator applies the physical force through the skin received in the hole.

7. The therapeutic compound delivery system of claim 1, wherein the hole has a diameter between 0.3 mm and 5 mm.

8. The therapeutic compound delivery system of claim 1, wherein the actuating piston moves approximately 1 mm between the first unactuated position and the second actuated position.

9. The therapeutic compound delivery system of claim 1, wherein the implantable pump does not include a battery disposed in the housing.

10. The therapeutic compound delivery system of claim 1, further comprising a septum fluidly connected to the reservoir, wherein the reservoir is refillable through the septum.

11. The therapeutic compound delivery system of claim 1, wherein the actuatable portion of the implantable pump comprises:
a flexible tube fluidly connected to the reservoir with a first check valve; wherein the first check valve allows flow from the reservoir the flexible tube; and
an outlet fluidly connected to the flexible tube with a second check valve, wherein the second check valve allows flow from the flexible tube to the outlet,
wherein the actuating piston is configured to apply force to the flexible tube when the actuating piston moves from the first unactuated position to the second actuated position to dispense the first volume of fluid through the outlet.

12. The therapeutic compound delivery system of claim 11, wherein movement of the actuating piston from the second actuated position to the first unactuated position allows the flexible tube to return to a relaxed state and draw a second volume of fluid from the reservoir into the flexible tube.

13. The therapeutic compound delivery system of claim 1, wherein the implantable pump is configured to sequentially dispense one of a plurality of fluid volumes each time the actuating piston is moved to the second actuated position, wherein each of the plurality of fluid volumes is within 10% of a target volume.

14. The therapeutic compound delivery system of claim 1, wherein the first volume includes bromocriptine dissolved in a combination of ethanol and Tween80.

15-50. (canceled)

51. A method of operating a therapeutic compound delivery system, the method comprising:
positioning an external actuator over skin under which an actuatable pump is disposed;
axially aligning an actuating piston disposed in the pump with a pusher of the external actuator; and
actuating the external actuator to depress the skin with the pusher to apply force to the actuating piston to operate the pump.

52. The method of claim 51, wherein actuating the actuator to depress the skin surface comprises deforming the skin into a hole formed in the housing of the pump.

53. The method of claim 52, wherein deforming the skin into the hole moves the actuating piston from a first unactuated position to a second actuated position.

54. The method of claim 53, wherein moving the actuating piston from the first unactuated positon to the second actuated position dispenses a volume from the pump.

55. The method of claim 51, wherein axially aligning the actuating piston and the pusher includes magnetically aligning the actuating piston and the pusher.

56. The method of claim 51, further comprising indicating that the actuating piston and the pusher are axially aligned.

57. The method of claim 56, wherein indicating that the actuating piston and positioning magnet are axially aligned comprises detecting a combined magnetic field generated by a first magnetic material of the external actuator and a second magnetic material of the pump.

58. The method of claim 51, further comprising determining that a threshold force was applied to the skin surface by the external actuator.

59. The method of claim 51, wherein actuating the external actuator comprises moving a driveshaft of the external actuator from a first unactuated position to a second actuated position to displace the pusher.

60. The method of claim 59, wherein the first unactuated position is a proximal driveshaft position and the second actuated position is a distal driveshaft position.

61. The method of claim 59, wherein moving the driveshaft from the first unactuated position to the second actuated position includes supplying power to a solenoid of the external actuator.

62. The method of claim 59, wherein moving the driveshaft from the first unactuated position to the second actuated position includes applying manual force to a trigger.

63. The method of claim 51, further comprising sequentially actuating the external actuator to dispense a plurality of fluid volumes from the pump, wherein each of the plurality of fluid volumes is within 10% of a target volume.
